(19) United States
(12) Patent Application Publication
(10) Pub. No.: US 2026/0195899 A1
(43) Pub. Date: Jul. 9, 2026

(54) METHOD OF MONITORING EYE FATIGUE, SMART GLASSES, AND STORAGE MEDIUM

(71) Applicants: Fu Tai Hua Industry (Shenzhen) Co., Ltd., Shenzhen (CN); HON HAI PRECISION INDUSTRY CO., LTD., New Taipei (TW)

(72) Inventors: Cheng-Ching Chien, New Taipei (TW); Xiang Huang, Shenzhen (CN)

(21) Appl. No.: 19/230,513

(22) Filed: Jun. 6, 2025

(30) Foreign Application Priority Data
Jan. 6, 2025 (CN) 202510026537.3

(51) Int. Cl. G06T 7/00; G06F 3/01; G06T 7/13; G06T 7/136; G06T 7/60

(52) U.S. Cl. CPC G06T 7/0016; G06F 3/013; G06T 7/13; G06T 7/136; G06T 7/60; G06T 2207/20061; G06T 2207/30041; G06T 2207/30232

(57) ABSTRACT

A method of monitoring eye fatigue is provided. In the method, smart glasses collect eye images of a user at a first moment and a second moment. The smart glasses determine a first size of a pupil in the eye image at the first moment, and a second size of a pupil in the eye image at the second moment. In response that an absolute difference between the second size and the first size is greater than a preset threshold, an eye image at a third moment can be collected. In response that an absolute difference between a third size of a pupil in the eye image at the third moment and the second size is greater than the preset threshold, the smart glasses determine that the eye of the user is in a fatigued state. The above method can improve the accuracy of eye fatigue monitoring.

### FIELD

The present disclosure relates to a field of image processing technology, and more particularly to a method of monitoring eye fatigue, smart glasses, and a storage medium.

### BACKGROUND

With the development of the Internet, an increasing number of users rely on terminal devices for work, study, and entertainment, resulting in prolonged focus on display screens. Prolonged focus on display screens may cause eye fatigue, thereby affecting visual health. Eye fatigue state is mainly evaluated based on blinking frequency. However, such method fails to detect eye fatigue conditions in a timely and accurate manner, as some users do not close their eyes for rest even when their eyes are already fatigued.

### BRIEF DESCRIPTION OF THE DRAWINGS

In order to more clearly illustrate technical solutions in the embodiments of the present disclosure, drawings that need to be used in the descriptions of the embodiments or the prior art will be briefly introduced below. Obviously, the drawings in the following description are merely some examples of the present disclosure, those of ordinary skill in the art can also obtain other drawings based on the drawings without paying creative efforts.

FIG. 1 is a schematic diagram of an application scenario of a method of monitoring eye fatigue in an embodiment of the present disclosure.

FIG. 2 is a flowchart of the method of monitoring eye fatigue in an embodiment of the present disclosure.

FIG. 3 is a schematic diagram of an image collection process in an embodiment of the present disclosure.

FIG. 4A is a schematic diagram of an eye image in an embodiment of the present disclosure.

FIG. 4B is a schematic diagram of a preprocessed image of the eye image shown in FIG. 4A in an embodiment of the present disclosure.

FIG. 4C is a schematic diagram of a plurality of feature points in the eye image shown in FIG. 4A after preprocessing in an embodiment of the present disclosure.

FIG. 4D is a schematic diagram of a target detection frame in the eye image shown in FIG. 4A after preprocessing in an embodiment of the present disclosure.

FIG. 5 is a flowchart of the method of monitoring eye fatigue in another embodiment of the present disclosure.

FIG. 6 is a flowchart of the method of monitoring eye fatigue in yet another embodiment of the present disclosure.

FIG. 7A is a schematic diagram of light-emitting points in a display screen in an embodiment of the present disclosure.

FIG. 7B is a schematic diagram of light-emitting devices in smart glasses in an embodiment of the present disclosure.

FIG. 8 is a structural diagram of a device for monitoring eye fatigue in an embodiment of the present disclosure.

FIG. 9 is a structural diagram of smart glasses in an embodiment of the present disclosure.

### DETAILED DESCRIPTION

The technical solutions in the embodiments of the present disclosure will be clearly and completely described below in conjunction with the drawings in the embodiments of the present disclosure, and it is obvious that a described embodiment is a part of the embodiments of the present disclosure, not all of the embodiments. Based on the embodiments in the present disclosure, all other embodiments obtained by a person of ordinary skill in the art without creative labor fall within the scope of protection of the present disclosure.

Hereinafter, terms “first” and “second” are used for descriptive purposes only, and are not to be construed as indicating or implying relative importance or implicitly specifying the number of technical features indicated. Thus, a feature defined with “first” or “second” may include one or more such features, either explicitly or implicitly. In the description of the embodiments of the present disclosure, words “exemplary” or “for example” are used to indicate an example, an illustration, or an illustration. Any embodiment or design solution described as “exemplary” or “for example” in the embodiments of the present disclosure should not be construed as being preferred or advantageous over other embodiments or design solutions. Rather, the words “exemplary” or “for example” is intended to present the relevant concepts in a specific manner.

Unless otherwise defined, all technical and scientific terms used herein have the same meaning as commonly understood by those skilled in the art in which this disclosure is filed. The terms used in the specification of this disclosure are used only for the purpose of describing specific embodiments and are not intended to limit this disclosure. It should be understood that in this disclosure, unless otherwise indicated, “/” means or. For example, A/B may denote either A or B. “And/or” in this disclosure is merely an associative relationship describing an associated object, indicating that three relationships may exist. For example, A and/or B can mean: A alone, both A and B, and B alone. “At least one” means one or more. “More than one” means two or more than two. For example, at least one of “a”, “b” or “c” can mean: “a”, “b”, “c”, “a” and “b”, “a” and “c”, b and “c”, “a”, “b” and “c” in seven cases.

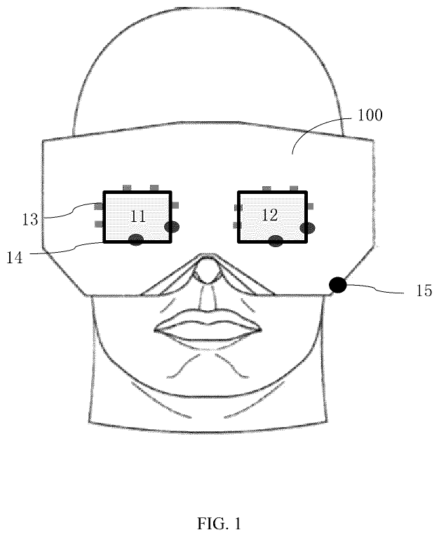

FIG. 1 is a schematic diagram of an application scenario of a method of monitoring eye fatigue in an embodiment of the present disclosure. As shown in FIG. 1, the method of monitoring eye fatigue can be applied to smart glasses 100. The smart glasses 100 include a first display screen 11, a second display screen 12, light-emitting devices 13, a camera 14, and an audio input/output module 15.

In some embodiments, the smart glasses 100 can be Augmented Reality (AR) glasses, Virtual Reality (VR) glasses or Mixed Reality (MR) glasses, and the smart glasses 100 can be applicable to entertainment, education, medical, and other fields. For example, in entertainment scenarios, the smart glasses 100 enable users to immerse themselves in gaming, movie watching, and other interactive activities. In the education field, the smart glasses 100 may be used as a learning tool that provides students with immersive learning environments via simulated experiments, reconstructing historical scenarios, and other pedagogical approaches. In the medical field, the smart glasses 100 may further support telemedicine, real-time diagnostics, and other clinical applications, thereby allowing doctors to acquire real-time patient data through the smart glasses.

The first display screen 11 can be configured at a first position of the smart glasses 100, the second display screen 12 can be configured at a second position of the smart glasses 100, and the first display screen 11 and/or the second display screen 12 may be configured to display a preset interface. Users can view the preset interface through the first display screen 11 and/or the second display screen 12. The preset interface can be an interface with light brightnesses that are less than a preset brightness. The preset interface can display rotating patterns of light-emitting points. In practical applications, the smart glasses 100 may include more or fewer display screens.

The light-emitting devices 13 can be Light Emitting Diodes (LEDs). The LEDs generate light through energy release via electron-hole recombination and are characterized by current-sensitive properties and extended operational longevity. A dense LED array may function as a light emitter by converting electrical data signals into optical signals, and may be applied to printing systems, display apparatuses, illumination devices, and other technical implementations.

The camera 14 can be an infrared camera. The light-emitting devices 13 and the camera 14 may be integrated to form an eye-tracking module of the smart glasses 100. The light-emitting devices 13 are configured to emit light at a preset wavelength, and the camera 14 can receive light emitted by the light-emitting devices 13, thereby capturing eye images.

The audio input/output module 15 can include microphones, speakers, etc. The audio input/output module 15 can be used to capture user voice commands, and recognize voice inputs. The smart glasses 100 can output voice prompt information to users, such as action instructions, monitoring results, etc.

It should be noted that the number of devices in FIG. 1 is only for illustrative purposes and does not constitute a limitation on the embodiments of the present disclosure.

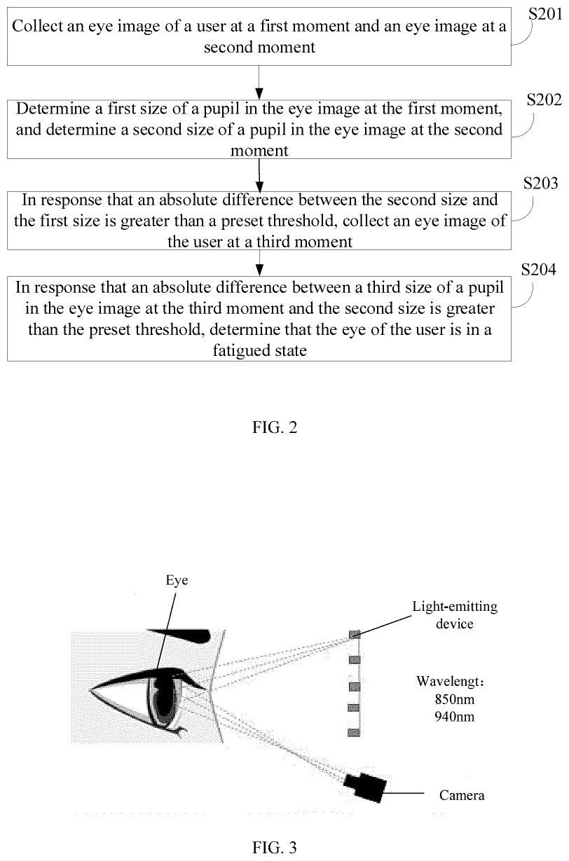

FIG. 2 is a flowchart of the method of monitoring eye fatigue in an embodiment of the present disclosure. As shown in FIG. 2, the method of monitoring eye fatigue in the embodiments of the present disclosure includes blocks S201-S204. According to different requirements, an order of the following blocks in the flowchart can be changed, and some of them can be omitted.

In block S201, the smart glasses collect an eye image of a user at a first moment and an eye image at a second moment.

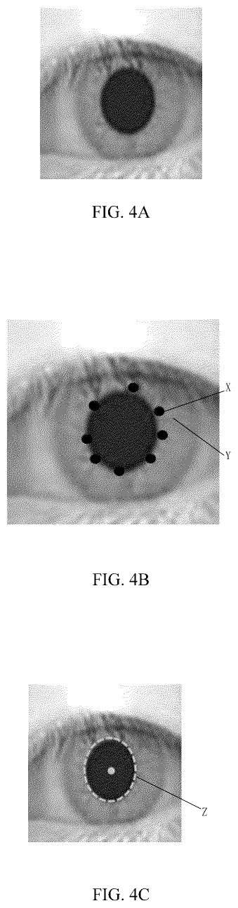

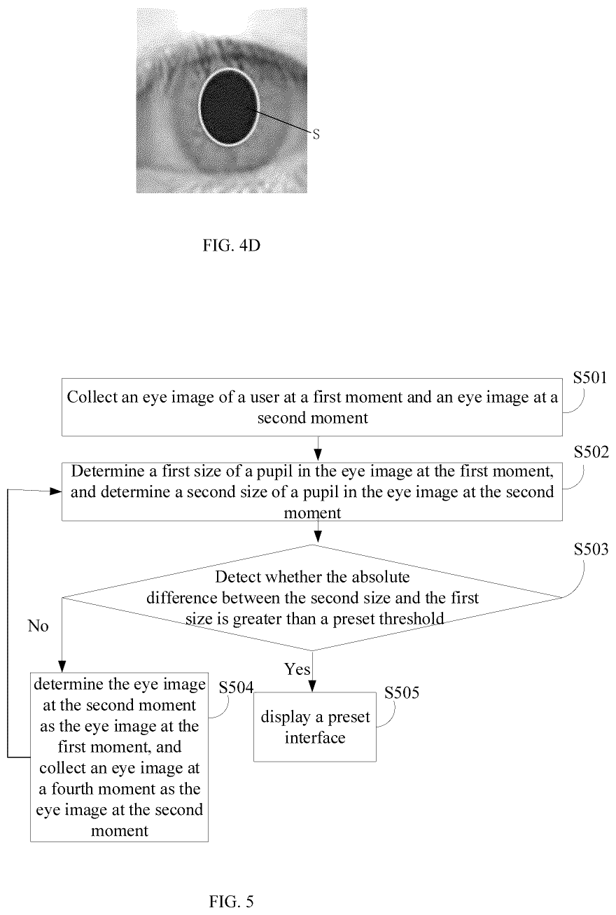

In one embodiment, in response that a user wears the smart glasses, the smart glasses can control the light-emitting devices to emit light with preset wavelength. The light with the preset wavelength may include but is not limited to infrared light with a wavelength of 850 nm, and infrared light with a wavelength of 940 nm. In response that the light emitted by the light-emitting devices illuminates eyes of a user, the smart glasses can control an infrared sensor integrated within the camera 14 to capture reflected light. The reflected light may include light reflected or light scattered back from the eyes of the user. The reflected light is converted into electrical signals by a photo detector of the infrared sensor via photoelectric conversion. The electrical signals are subsequently transformed into digital image data through a signal processing module of the infrared sensor, thereby generating an eye image. For illustrative clarity, please refer to FIG. 3 and FIG. 4A, the light-emitting devices can emit infrared light of 850 nm and infrared light of 940 nm toward the eyes for acquiring pupil feature. The pupil and area surrounding the pupil can reflect or scatter a portion of the light, and the camera can capture the eye image as shown in FIG. 4A.

The eye image is collected by emitting light with the preset wavelength. Since the preset wavelength light is less susceptible to interference from surrounding environment, clear eye images can be steadily collected even in dark or bright light environments, thereby improving flexibility and reliability of image collection. Furthermore, since the preset wavelength light can penetrate obstacles (such as gauze, eyeglass lenses, etc.), the light with the preset wavelength can eliminate the influence of obstacles on eye image collection, thus the accuracy and stability of image collection can be improved.

In one embodiment, the first moment is earlier than the second moment, and a time interval between the second moment and the first moment can be a first preset duration. The first preset duration can be set according to actual requirements. For example, the first preset duration can be set as 30 minutes. In response that the first moment is 10:00, the second moment can be 10:30.

In block S202, the smart glasses determine a first size of a pupil in the eye image at the first moment, and determine a second size of a pupil in the eye image at the second moment.

In one embodiment, the smart glasses acquire a preprocessed image by pre-processing the eye image at the first moment, and determine a plurality of feature points by performing an edge detection on the preprocessed image. The smart glasses determine a target detection frame based on the plurality of feature points, and calculate the first size based on the target detection frame. The first size can be represented as a size of the pupil in the eye image, for example, the first size can include diameter of the pupil in the eye image, and radius of the pupil in the eye image.

By preprocessing, the noises in the eye image can be removed, thereby reducing the data volume of pixels in the eye image and improving calculation efficiency of the first size. Additionally, since the noises in the eye image are removed, the image quality can be improved, thereby improving the accuracy of the target detection frame, and consequently improving a calculation accuracy of the first size.

In one embodiment, the smart glasses generate a grayscale image by performing a grayscale processing on the eye image at the first moment. The smart glasses remove noises from the grayscale image by using a preset filter, and obtain a filtered image. The smart glasses perform a binarization processing on the filtered image, and obtain the preprocessed image. The preset filter can be determined according to types of noises.

The color information in the eye image can be eliminated via the grayscale processing, thereby reducing computational complexity. By utilizing the preset filter, the noises in the grayscale image can be effectively reduced, thereby improving image clarity and quality. Through the binarization processing of the filtered image, a distinction between foreground and background in the filtered image becomes more obvious.

In one embodiment, the eye image can be stored in Red-Green-Blue (RGB). Each pixel in the eye image can include three color channels. The three color channels can include a Red (R) channel, a Green (G) channel, and a Blue (B) channel. The three color channels in the eye image are separated, and brightness values of each pixel in the three color channels are obtained.

In a process of generating the grayscale image, the smart glasses determine a grayscale value of each pixel in the eye image at the first moment, and generate the grayscale image based on the grayscale value of each pixel in the eye image at the first moment. A formula for determining the grayscale value can be represent as: y=k1×R+k2×G+k3×B, in which “y” represents the grayscale value of each pixel, “R” represents a brightness value of each pixel in the red channel, “G” represents a brightness value of each pixel in the green channel, B″ represents a brightness value of each pixel in the blue channel, “k1” represents a weight corresponding to the brightness value of each pixel in the red channel, “k2” represents a weight corresponding to the brightness value of each pixel in the green channel, and “k3” represents a weight corresponding to the brightness value of each pixel in the blue channel. For example, it is assumed that “k1” is 0.299, “k2” is 0.587, “k3” is 0.114, and a pixel “A” has a red brightness value “R”, a green brightness value “G”, and a blue brightness value “B”, the grayscale value of the pixel “A” can be represent as: 0.299×R+0.587×G+0.114×B.

The smart glasses perform the grayscale processing on the eye image at the first moment, thereby eliminating color information and retaining only luminance data. This grayscale conversion significantly reduces the data volume of the grayscale image.

In a process of obtaining the filtered image, the smart glasses analyze noise distribution characteristics of the grayscale image by using statistical methods. The smart glasses determine a noise type of the grayscale image based on the noise distribution characteristics, and utilize the preset filter corresponding to the noise type. The smart glasses perform a removing processing on the noises in the grayscale image, and obtain the filtered image.

In a process of determining the noise type of the grayscale image, the smart glasses count a number of occurrences of each grayscale value in the grayscale image. The smart glasses construct a grayscale histogram by using the grayscale values as a horizontal axis and using the number of occurrences of each grayscale value in the grayscale image as a vertical axis. The smart glasses determine the noise type based on the grayscale histogram. For example, a grayscale histogram corresponding to Gaussian noise type exhibits a Gaussian distribution, a grayscale histogram corresponding to salt-and-pepper noise type exhibits spikes near a minimum grayscale value and a maximum grayscale value.

The smart glasses determine the noise type and utilize the preset filter corresponding to the noise type to perform a denoising processing on the noises in the grayscale image, thereby accurately eliminating the noises in the grayscale image and improving the quality of the grayscale image.

In a process of obtaining the preprocessed image, the smart glasses divide the filtered image into a plurality of sub-regions, calculate a median pixel value of all pixel values in each of the sub-regions and determine the median pixel value corresponding to each of the sub-regions as a local threshold for each of the sub-regions. The smart glasses compare a pixel value of each pixel in each of the sub-regions with a corresponding local threshold, update pixel values to be a first value in response that pixel values are greater than or equal to the local threshold, or update pixel values to be a second value in response that the pixel values are less than the local threshold, and obtain the preprocessed image. The first value is greater than the second value, for example, the first value can be set as 255, and the second value can be set as 0. For better understanding, please refer to FIG. 4B, FIG. 4B is a schematic diagram of a preprocessed image of the eye image shown in FIG. 4A in an embodiment of the present disclosure. As shown in FIG. 4B, pixel “X” can represent a pixel whose pixel value is equal to the first value, and pixel “Y” can represent a pixel whose pixel value is equal to the second value.

The smart glasses can reduce the image information in the preprocessed image through the binarization processing, thereby improving the efficiency of edge detection.

In a process of determining the plurality of feature points, the smart glasses determine a neighborhood for each first pixel in the preprocessed image, determine an absolute difference in pixel values between each first pixel and each second pixel within a corresponding neighborhood of each first pixel. In response that an absolute difference is greater than a preset difference, the smart glasses determine second pixels whose corresponding absolute differences are greater than the preset difference, as the plurality of feature points.

A size and a shape of the neighborhood may be modified according to actual situations. Please refer to FIG. 4C, FIG. 4C is a schematic diagram of a plurality of feature points in the eye image shown in FIG. 4A after preprocessing in an embodiment of the present disclosure. As shown in FIG. 4C, pixel “Z” is represented as a feature point. For example, the pixel “Z” can include a pixel “X” in FIG. 4B, and a pixel “Y” that is adjacent to the pixel “X”.

The smart glasses can determine pixels whose pixel values are suddenly changed by analyzing each first pixel and the corresponding neighborhood, thereby improving the accuracy of the feature points.

In a process of determining the target detection frame, the smart glasses determine feature points having same pixel values as target feature points, and calculate a distance between any two target feature points. The smart glasses generate a plurality of first detection frames by connecting two selected target feature points from the determined feature points, a distance between the two selected target feature points is smaller than a preset distance threshold. The smart glasses determine a plurality of second detection frames from the plurality of first detection frames, and calculate shape similarities between the plurality of second detection frames and a preset shape. The smart glasses determine the target detection frame from the plurality of second detection frames, the target detection frame corresponds to the highest shape similarity from the calculated shape similarities.

In a process of generating the plurality of first detection frames, the smart glasses select a feature point from the feature points randomly as a tracking point, and search a neighborhood of the tracking point for feature points that satisfy preset conditions. The smart glasses determine the feature points that satisfy the preset conditions as updated tracking points, and marking status of found feature points that satisfy the preset conditions to be visited status, until all feature points that satisfy the preset conditions have been found. The smart glasses connect the found feature points that satisfy the preset conditions, and obtain the first detection frames. The preset conditions may include but are not limited to feature points having a same pixel value as the tracking point, feature points with a distance from the tracking point less than a preset distance threshold. The preset distance threshold can be set and adjusted based on empirical values.

In a process of determining the plurality of second detection frames, the smart glasses set parameters for Hough Circle Transform. The smart glasses perform the Hough Circle Transform on the first detection frames based on the parameters, and obtain position information of target point corresponding to each of the first detection frames and a first distance corresponding to each of the first detection frames. Based on the position information of the target point, the first distance, and a preset direction, the smart glasses determine the position information of prediction points, distances between each of the prediction points and the target point are equal to the first distance. For each of the first detection frames, the smart glasses count a number of prediction points that are overlapped with edge points in each of the first detection frames, based on the position information of the prediction points. The smart glasses determine first detection frames corresponding to a number greater than the preset threshold, as the second detection frames. The preset direction and the preset threshold can be set and adjusted according to requirements. A formula for determining the position information of target point can be represented as: (x−a)²+(y−b)²=r², where “(x,y)” represents the position information of the edge points in each of the first detection frames, “(a,b)” represents the position information of the target point, and “r” represents the first distance.

The smart glasses preliminarily determine the second detection frames that satisfy conditions from the first detection frames, it can reduce the number of the second detection frames, thereby improving the efficiency of determining the target detection frame.

In a process of calculating the shape similarity, the smart glasses calculate a Euclidean distance between the target point and a preset point in the preset shape, based on the position information of the target point and position information of the preset point. The formula for determining the Euclidean distance can be represented as: d=\sqrt{(a−m)²+(b−n)²}, where “(a,b)” represents the position information of the target point, “(m,n)” represents the position information of the preset point. The smart glasses perform the Hough Circle Transform on the preset shape and obtain a second distance corresponding to the preset shape. The smart glasses calculate an absolute difference between the first distance and the second distance. The smart glasses calculate the shape similarity between each of the second detection frames and the preset shape based on the Euclidean distance, the absolute difference, a first threshold, and a second threshold. The formula for determining the shape similarity can be represented as:

$$S = 1 - ( \frac{d}{{T}_{d} + c} + \frac{R}{{T}_{r} + c} ) ,$$

in which “S” represents the shape similarity between each of the second detection frames and the preset shape, “d” represents the Euclidean distance between the target point and the preset point, “R” represents the absolute difference between the first distance and the second distance, “T_{d}” represents the first threshold, “Tᵣ” represents the second threshold, and “c” is a preset positive number. “T_{d}”, “Tᵣ”, and) “c” can be set and adjusted according to actual situations.

The smart glasses can accurately calculate the shape similarity between each of the second detection frames and the preset shape by combining the Euclidean distance and the absolute difference. The Euclidean distance represents a distance between the target point and the preset point, and the absolute difference represents a distance between the first distance and the second distance. Accordingly, the accuracy of the target detection frame can be improved.

In some other embodiments, the smart glasses generate a plurality of third detection frames based on the plurality of feature points through the Hough Circle Transform. The smart glasses select the target detection frame from the plurality of third detection frames based on preset filtering criteria.

In a process of obtaining the third detection frames, the smart glasses set parameters for the Hough Circle Transform. The smart glasses perform the Hough Circle Transform on each of the feature points based on the parameters for the Hough Circle Transform, and obtain shape information of a detection shape corresponding to each of the feature points. The smart glasses count a number of detection shapes having same shape information, and determine the third detection frames according to the detection shapes whose numbers are greater than a threshold.

The preset filtering criteria may include but are not limited to a contour size, a contour shape, and a contour position in the eye image. For example, the target detection frame can be a pupil contour that is located at a center position of the eye image, and a shape of the target detection frame can be close to a circle. For better understanding, please refer to FIG. 4D, FIG. 4D is a schematic diagram of a target detection frame in the eye image shown in FIG. 4A after preprocessing in an embodiment of the present disclosure. As shown in FIG. 4D, shape “S” can be represented as the target detection frame.

In this embodiment, by performing the Hough Circle Transform on the feature points, and the third detection frames are obtained. The detection efficiency is improved since there is no need to process an entire eye image. Additionally, by filtering the third detection frames, the accuracy of the target detection frame is enhanced.

In some embodiments, the smart glasses determine parameter information of the target detection frame, and calculate the first size based on the parameter information. The parameter information may include the first distance.

A formula for calculating the first size can be represented as: d=k×r, where “d” represents the first size, “r” represents the first distance corresponding to the target detection frame, “k” can be set to be 2 or other values.

In some embodiments, a method for determining the second size is similar to a method for determining the first size, it is not repeatedly described here.

In block S203, in response that an absolute difference between the second size and the first size is greater than a preset threshold, the smart glasses collect an eye image of the user at a third moment.

In one embodiment, the preset threshold can be set according to actual requirements. For example, the preset threshold can be set to be 0.3 mm. In response that the absolute difference between the second size and the first size is greater than 0.3 mm, the smart glasses determine that the eye of the user may be in a fatigue state. In order to more accurately determine whether the pupil is in a fatigue state and filter out other factors that affect pupil size, the eye image can be collected at the third moment to improve the accuracy of determining eye state.

In one embodiment, the third moment is later than the second moment, and a time interval between the third moment and the second moment can be a second preset duration, the second preset duration can be set according to actual requirements. The second preset duration can be shorter than the first preset duration. In the example as mentioned above, in response that the first preset duration is set to be 30 minutes, the second preset duration can be set to be 1 minute. For example, the second moment is 10:30, the third moment can be 10:31.

A collection method for the eye image at the third moment is similar to a collection method for the eye image at the first moment, it is not repeated here.

In block S204, in response that an absolute difference between a third size of a pupil in the eye image at the third moment and the second size is greater than the preset threshold, the smart glasses determine that the eye of the user is in a fatigued state.

In one embodiment, in response that the absolute difference between the third size of the pupil in the eye image at the third moment and the second size is less than or equal to the preset threshold, the smart glasses determine that the eye of the user is not in a fatigued state.

For example, the absolute difference between the third size and the second size is greater than 0.3 mm, the smart glasses determine that the eye of the user is in a fatigued state. For another example, the absolute difference between the third size and the second size is less than or equal to 0.3 mm, the smart glasses determine that the eye of the user is not in a fatigued state.

By determining the absolute difference between the second size and the third size, and comparing the absolute difference with the preset threshold, the accuracy of eye fatigue monitoring can be improved through combining comparisons of pupil size changes with the preset threshold.

In the embodiments of the method of monitoring eye fatigue, by determining the absolute difference between the first size of the pupil in the eye image at the first moment and the second size of the pupil in the eye image at the second moment, comparing the absolute difference with the preset threshold, it can preliminarily determine the fatigue state of the eyes of the user without monitoring through blinking frequency. Additionally, combined with comparing the absolute difference between the second size and the third size of the pupil in the eye image at the third moment with the preset threshold, it can accurately determine whether the eyes of the user are in a fatigued state, thus, it is beneficial for taking certain measures to relieve eye fatigue when the eyes of the user are in a fatigued state, thereby ensuring the user's eye health.

FIG. 5 is a flowchart of a method of monitoring eye fatigue in another embodiment of the present disclosure. As shown in FIG. 5, the method of monitoring eye fatigue in the embodiments of the present disclosure includes blocks S501-S505. According to different needs, an order of the following blocks in the flowchart can be changed, and some of them can be omitted.

In block S501, the smart glasses collect an eye image of a user at a first moment and an eye image at a second moment.

In block S502, the smart glasses determine a first size of a pupil in the eye image at the first moment, and determine a second size of a pupil in the eye image at the second moment.

The detailed content of blocks S501-S502 can refer to the detailed descriptions of blocks S201-S202 in FIG. 2 as mentioned above, it is not repeated here.

In block S503, the smart glasses detect whether the absolute difference between the second size and the first size is greater than a preset threshold.

In some embodiments, in response that the absolute difference between the second size and the first size is less than or equal to the preset threshold, the procedure goes to block S504. In response that the absolute difference between the second size and the first size is greater than the preset threshold, the procedure goes to block S505.

In block S504, the smart glasses determine the eye image at the second moment as the eye image at the first moment, and collect an eye image at a fourth moment as the eye image at the second moment.

In some embodiments, the fourth moment is subsequent to the second moment, and a time interval between the fourth moment and the second moment can be the first preset duration. For example, the second moment is 10:30, the fourth moment is 11:00.

In some embodiments, the procedure goes to block S502, after block S504.

S505, the smart glasses display a preset interface.

In some embodiments, the preset interface is configured to display within a field of view of the user. The preset interface may display with light brightness that are less than a preset brightness, The preset interface also may display rotating patterns of light-emitting points. For example, the smart glasses display a green screen with brightness that are less than the preset brightness.

Since pupil changes may also be caused by other factors, to filter out such situations, in response that the absolute difference between the second size and the first size is greater than the preset threshold, the smart glasses display the preset interface on the first display screen and/or the second display screen. By displaying the preset interface, it can avoid large changes in pupil size caused by factors such as strong light, thereby improving the reliability of eye fatigue monitoring.

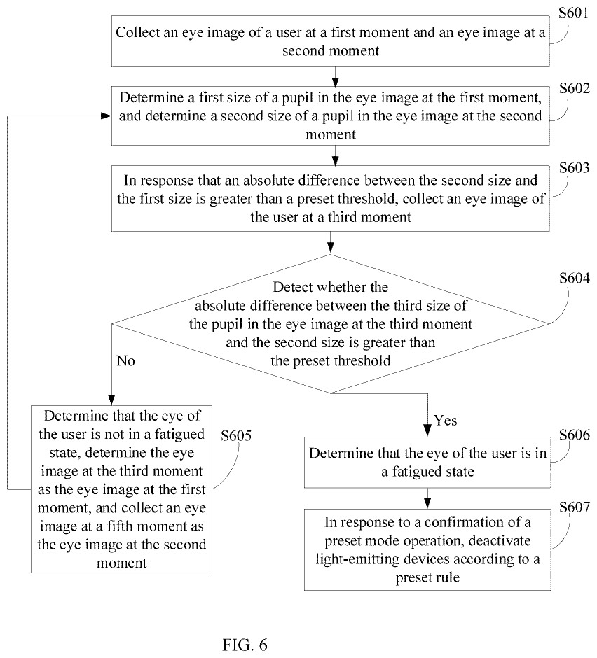

FIG. 6 is a flowchart of a method of monitoring eye fatigue in yet another embodiment of the present disclosure. As shown in FIG. 6, the method of monitoring eye fatigue in the embodiments of the present disclosure includes blocks S601-S606. According to different needs, an order of the following blocks in the flowchart can be changed, and some of them can be omitted.

In block S601, the smart glasses collect an eye image of a user at a first moment and an eye image at a second moment.

In block S602, the smart glasses determine a first size of a pupil in the eye image at the first moment, and determine a second size of a pupil in the eye image at the second moment.

In block S603, in response that an absolute difference between the second size and the first size is greater than a preset threshold, the smart glasses collect an eye image of the user at a third moment.

The detailed content of blocks S601-S603 can refer to the detailed descriptions of blocks S201-S203 in FIG. 2 as mentioned above, it is not repeated here.

In block S604, the smart glasses detect whether the absolute difference between the third size of the pupil in the eye image at the third moment and the second size is greater than the preset threshold.

In some embodiments, in response that the absolute difference between the second size and the first size is less than or equal to the preset threshold, the procedure goes to block S605. In response that the absolute difference between the third size and the second size is greater than the preset threshold, the procedure goes to block S606.

In block S605, the smart glasses determine that the eye of the user is not in a fatigued state.

In some embodiments, after determining that the eye of the user is not in a fatigued state, the smart glasses determine the eye image at the third moment as the eye image at the first moment, and collect an eye image at a fifth moment as the eye image at the second moment.

In some embodiments, the fifth moment is subsequent to the third moment, and a time interval between the fifth moment and the third moment can be the first preset duration. For example, the third time moment is 10:31, the fourth moment can be 11:01.

In some embodiments, the procedure goes to block S602 after completing block S605.

In block S606, the smart glasses determine that the eye of the user is in a fatigued state.

In some embodiments, the procedure goes to block S607 after completing block S606.

In block S607, in response to a confirmation of a preset mode operation, the smart glasses deactivate light-emitting devices according to a preset rule.

In one embodiment, the preset rule is configured to indicate a rotational direction of the eye of the user. The preset mode operation may include a first mode operation and a second mode operation, with different preset rules corresponding to different preset mode operations.

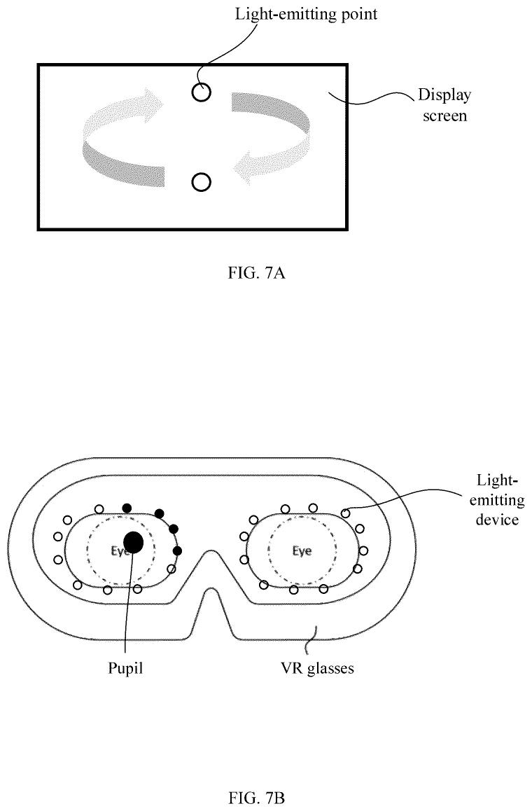

In one embodiment, in response to a confirmation of the first mode operation, light-emitting points are displayed on display screens (for example, the first display screen, the second display screen), and the light-emitting points are moved according to the preset rule. Based on display position of the light-emitting points on the display screen, the light-emitting devices corresponding to the display position are determined and turned off. The light-emitting devices corresponding to the display position can be LEDs. Combined with a movement of light-emitting points under the first mode operation in FIG. 7A, for example, as shown in FIG. 7A, the light-emitting points can rotate clockwise on the display screen based on the preset rule. Combined with FIG. 7B to explain a turning off of the light-emitting devices, as shown in FIG. 7B, in response that the light-emitting devices are turned on, the light-emitting devices correspond to hollow circles. In response that the light-emitting devices are turned off, the light-emitting devices correspond to solid circles. In response that displaying light-emitting points on the display screen in FIG. 7A, the pupils of the user can follow the rotation of the light-emitting points, and the light-emitting devices set in the corresponding areas can be turned off, shown as solid circles in FIG. 7B. In response that the pupils of the user follow the light-emitting points, the light-emitting devices in corresponding areas are turned off, thereby avoiding direct eye exposure to light and relieving eye fatigue.

In one embodiment, in response to a confirmation of the second mode operation, the light-emitting devices corresponding to an eye rotation direction are turned off, based on the rotation direction of the eye of the user.

By turning off the light-emitting devices in the direct line of sight, it can avoid direct eye exposure to light, thereby relieving the eye fatigue problem and ensuring eye health.

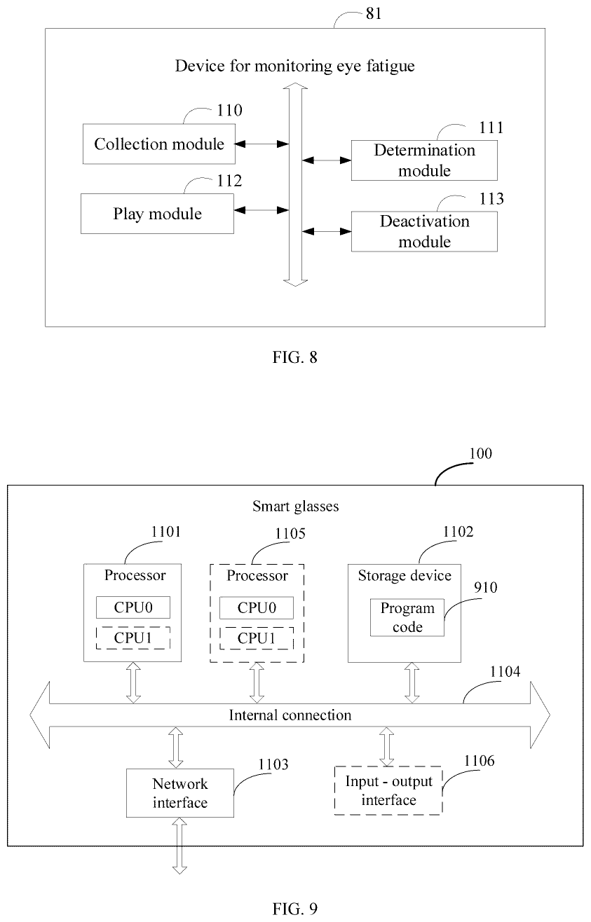

FIG. 8 is a structural diagram of a device for monitoring eye fatigue in an embodiment of the present disclosure. In some embodiments, the device for monitoring eye fatigue 81 may include a collection module 110, a determination module 111, a play module 112, and a deactivation module 113. The modules in this disclosure refers to a series of computer-readable instruction segments capable of being acquired by a processor (e.g., a processor 1101 shown in FIG. 9) and capable of accomplishing a fixed function, which are stored in a storage device (e.g., a storage device 1102 shown in FIG. 9).

The collection module 110 collects an eye image of a user at a first moment and an eye image at a second moment, the first moment is precedes the second moment. The determination module 111 determines a first size of a pupil in the eye image at the first moment, and determines a second size of a pupil in the eye image at the second moment. In response that an absolute difference between the second size and the first size is greater than a preset threshold, the collection module 110 collects an eye image of the user at a third moment, the third moment is subsequent to the second moment. In response that an absolute difference between a third size of a pupil in the eye image at the third moment and the second size is greater than the preset threshold, the determination module 111 determines that the eye of the user is in a fatigued state.

The determination module 111 acquires a preprocessed image by pre-processing the eye image at the first moment. The determination module 111 determines a plurality of feature points by performing an edge detection on the preprocessed image. The determination module 111 determines a target detection frame based on the plurality of feature points, and calculate the first size based on the target detection frame.

The determination module 111 generates a grayscale image by performing a grayscale processing on the eye image at the first moment. The determination module 111 removes noises from the grayscale image by using a preset filter, and obtains a filtered image. The determination module 111 performs a binarization processing on the filtered image, and obtains the preprocessed image.

The determination module 111 determines feature points having same pixel values as target feature points, and calculates a distance between any two target feature points. The determination module 111 generates a plurality of first detection frames by connecting two selected target feature points, a distance between the two selected target feature points being smaller than a preset distance threshold. The determination module 111 determines a plurality of second detection frames from the plurality of first detection frames, and calculates a shape similarity between each of the plurality of second detection frames and a preset shape. The determination module 111 determines the target detection frame from the plurality of second detection frames, with the target detection frame corresponding to a highest shape similarity.

The determination module 111 generates a plurality of third detection frames based on the plurality of feature points through a Hough Circle Transform. The determination module 111 selects the target detection frame from the plurality of third detection frames based on preset filtering criteria.

The determination module 111 determines parameter information of the target detection frame, and calculates the first size based on the parameter information.

The play module 112 displays a preset interface, which is presented within a field of view of the user.

In response to a confirmation of a preset mode operation, the deactivation module 113 deactivates light-emitting devices according to a preset rule, which defines a rotational direction of the eye of the user.

In the method of monitoring eye fatigue of these embodiments, by determining the absolute difference between the first size of the pupil in the eye image at the first moment and the second size of the pupil in the eye image at the second moment, comparing the absolute difference with the preset threshold, it can preliminarily determine the fatigue state of the eye of the user without monitoring through blinking frequency. Meanwhile, combined with comparing the absolute difference between the second size and the third size of the pupil in the eye image at the third moment with the preset threshold, it can accurately determine whether the eye of the user is in a fatigued state, which is beneficial for taking certain measures to relieve eye fatigue when the eye of the user is in a fatigued state, ensuring the user's eye health.

FIG. 9 is a structural diagram of smart glasses in an embodiment of the present disclosure. Optionally, the smart glasses 100 shown in FIG. 9 are used to execute the methods illustrated in FIG. 2, FIG. 5 and FIG. 6.

The smart glasses 100 include at least one processor 1101, a storage device 1102, and at least one network interface 1103.

For example, the processor 1101 may be a general-purpose central processing unit (CPU), a network processor (NP), a graphics processing unit (GPU), a neural-network processing unit (NPU), a data processing unit (DPU), a microprocessor, or one or more integrated circuits for implementing the solutions of the present disclosure. For example, the processor 1101 includes an application-specific integrated circuit (ASIC), a programmable logic device (PLD), or a combination thereof. For example, the PLD may be a complex programmable logic device (CPLD), a field-programmable gate array (FPGA), a generic array logic (GAL), or any combination thereof.

The storage device 1102 may be a read-only memory (ROM) or another type of static storage device for storing static information and instructions, or a random access memory (RAM) or another type of dynamic storage device for storing information and instructions. It may also be an electrically erasable programmable read-only memory (EEPROM), compact disc read-only memory (CD-ROM), other optical disc storage (including compact discs, laser discs, optical discs, digital versatile discs, Blu-ray discs, etc.), magnetic disk storage media, other magnetic storage devices, or any other medium capable of carrying or storing program code in the form of instructions or data structures and accessible by a computer, though not limited to these options. Optionally, the storage device 1102 exists independently and is connected to the processor 1101 via an internal connection 1104. Alternatively, the storage device 1102 and processor 1101 may be integrated.

The network interface 1103 employs any transceiver-like device for communicating with other devices or communication networks. It may include at least one of a wired network interface or a wireless network interface. The wired network interface could be an Ethernet interface (e.g., an optical interface, electrical interface, or a combination). The wireless network interface might be a wireless local area network (WLAN) interface, cellular network interface, or a combination thereof.

In some embodiments, the processor 1101 includes one or more CPUs, such as CPU0 and CPU1 shown in FIG. 9.

In some embodiments, the smart glasses 100 include multiple processors, such as the processor 1101 and the processor 1105 shown in FIG. 9. Each of these processors may be a single-core processor (single-CPU) or a multi-core processor (multi-CPU). Here, a processor optionally refers to one or more devices, circuits, and/or processing cores for processing data (such as computer program instructions).

In some embodiments, the smart glasses 100 also include an internal connection 1104. The processor 1101, the storage device 1102, and at least one network interface 1103 are connected via the internal connection 1104. The internal connection 1104 includes pathways for transmitting information between the above-mentioned components. Optionally, the internal connection 1104 is a single board or a bus. Optionally, the internal connection 1104 is divided into an address bus, a data bus, a control bus, etc.

In some embodiments, the smart glasses 100 also include an input-output interface 1106. The input-output interface 1106 is connected to the internal connection 1104.

Optionally, the processor 1101 implements the methods in the above-mentioned embodiments by reading the program code 910 stored in the storage device 1102, or the processor 1101 implements the methods in the above-mentioned embodiments by the program code stored internally. In the case where the processor 1101 implements the methods in the above-mentioned embodiments by reading the program code 910 stored in the storage device 1102, the storage device 1102 stores the program code for implementing the methods provided by the embodiments of the present disclosure.

For further details on how the processor 1101 implements the above functions, refer to the descriptions in the preceding method embodiments, which will not be repeated here.

This embodiment also provides a computer storage medium storing computer instructions. In response that the computer instructions are executed by the smart glasses, the smart glasses perform the relevant method steps and implement the method of monitoring eye fatigue described in the embodiments.

Additionally, an embodiment of this disclosure provides a device, which may be a chip, component, or module. The device includes a connected processor and memory, where the memory stores computer-executable instructions. When the device operates, the processor executes the instructions to enable the chip to perform the method of monitoring eye fatigue described in the foregoing method embodiments.

The smart glasses, computer storage medium, computer program product, and chip provided in this embodiment are all configured to execute the corresponding methods described above. Therefore, their beneficial effects align with those of the corresponding methods and will not be reiterated here.

Through the description of the above implementation methods, those skilled in the art can clearly understand that, for the convenience and conciseness of description, the above division of functional modules is only used as an example. In practical applications, the above functions can be allocated to be completed by different functional modules according to needs, that is, the internal structure of the device can be divided into different functional modules to complete all or part of the functions described above.

In the several embodiments provided in the present disclosure, it should be understood that the disclosed devices and methods can be implemented in other ways. For example, the above-described device embodiments are only illustrative. For instance, the division of modules or units is merely a logical functional division, and there can be other division methods in actual implementation. For example, multiple units or components can be combined or integrated into another device, or some features can be ignored or not executed. Furthermore, the shown or discussed coupling or direct coupling or communication connection between components can be through some interfaces, and indirect coupling or communication connection between devices or units can be electrical, mechanical, or in other forms.

The units described as separate components may or may not be physically separate. The components shown as units may be one physical unit or multiple physical units, that is, they can be located in one place or distributed to multiple different places. Some or all units can be selected to achieve the purpose of the embodiment solution according to actual needs.

Additionally, in various embodiments of the present disclosure, the functional units can be integrated into one processing unit, or can exist physically separately, or two or more units can be integrated into one unit. The above integrated units can be implemented in the form of hardware or in the form of software functional units.

When the integrated unit is implemented in the form of a software functional unit and sold or used as an independent product, it can be stored in a readable storage medium. Based on such understanding, the technical solution of the embodiments of the present application, or the part that makes contributions to the prior art, or all or part of the technical solution can be embodied in the form of a software product. The software product is stored in a storage medium and includes several instructions for enabling a device (which can be a microcontroller, chip, etc.) or processor to execute all or part of the steps of the methods in various embodiments of the present application. The aforementioned storage medium includes: USB flash drives, mobile hard drives, Read-Only Memory (ROM), Random Access Memory (RAM), magnetic disks or optical discs, and various media that can store program code.

The above are only specific implementation methods of the present disclosure, but the protection scope of the present disclosure is not limited to this. Any changes or replacements within the technical scope disclosed in the present disclosure should be covered within the protection scope of the present disclosure. Therefore, the protection scope of the present disclosure should be subject to the protection scope of the claims.

What is claimed is:

1. A method of monitoring eye fatigue, the method comprising:
collecting an eye image of a user at a first moment and an eye image at a second moment, wherein the first moment is earlier than the second moment;
determining a first size of a pupil in the eye image at the first moment, and determining a second size of a pupil in the eye image at the second moment;
in response that an absolute difference between the second size and the first size is greater than a preset threshold, collecting an eye image of the user at a third moment, wherein the third moment is later than the second moment; and
in response that an absolute difference between a third size of a pupil in the eye image at the third moment and the second size is greater than the preset threshold, determining that the eye of the user is in a fatigued state.

2. The method of claim 1, wherein determining the first size of the pupil in the eye image at the first moment comprises:
acquiring a preprocessed image by pre-processing the eye image at the first moment;
determining a plurality of feature points by performing an edge detection on the preprocessed image;
determining a target detection frame based on the plurality of feature points; and
calculating the first size based on the target detection frame.

3. The method of claim 2, wherein acquiring the preprocessed image by pre-processing the eye image at the first moment comprises:
generating a grayscale image by performing a grayscale processing on the eye image at the first moment;
removing noises from the grayscale image by using a preset filter, and obtaining a filtered image;
performing a binarization processing on the filtered image, and obtaining the preprocessed image.

4. The method of claim 2, wherein determining the target detection frame based on the plurality of feature points comprises:
determining feature points having same pixel values as target feature points, and calculating a distance between any two target feature points;
generating a plurality of first detection frames by connecting two selected target feature points from the determined feature points, wherein a distance between the two selected target feature points is smaller than a preset distance threshold;
determining a plurality of second detection frames from the plurality of first detection frames;
calculating shape similarities between the plurality of second detection frames and a preset shape;
determining the target detection frame from the plurality of second detection frames, with the target detection frame corresponding to a highest shape similarity from the calculated shape similarities.

5. The method of claim 2, wherein determining the target detection frame based on the plurality of feature points comprises:
generating a plurality of third detection frames based on the plurality of feature points through a Hough Circle Transform;
selecting the target detection frame from the plurality of third detection frames based on preset filtering criteria.

6. The method of claim 2, wherein calculating the first size based on the target detection frame comprises:
determining parameter information of the target detection frame;
calculating the first size based on the parameter information.

7. The method of claim 1, further comprising:
displaying a preset interface presented within a field of view of the user.

8. The method of claim 1, further comprising:
in response to a confirmation of a preset mode operation, deactivating light-emitting devices according to a preset rule, the preset rule defines a rotational direction of the eye of the user.

9. Smart glasses comprising:
a processor; and
a storage device storing a plurality of instructions, which when executed by the processor, cause the processor to:
collect an eye image of a user at a first moment and an eye image at a second moment, wherein the first moment is earlier than the second moment;
determine a first size of a pupil in the eye image at the first moment, and determine a second size of a pupil in the eye image at the second moment;
in response that an absolute difference between the second size and the first size is greater than a preset threshold, collect an eye image of the user at a third moment, wherein the third moment is later than the second moment; and
in response that an absolute difference between a third size of a pupil in the eye image at the third moment and the second size is greater than the preset threshold, determine that the eye of the user is in a fatigued state.

10. The electronic device of claim 9, wherein the processor is further caused to:
acquire a preprocessed image by pre-processing the eye image at the first moment;
determine a plurality of feature points by performing an edge detection on the preprocessed image;
determine a target detection frame based on the plurality of feature points; and
calculate the first size based on the target detection frame.

11. The electronic device of claim 10, wherein the processor is further caused to:
generate a grayscale image by performing a grayscale processing on the eye image at the first moment;
remove noises from the grayscale image by using a preset filter, and obtain a filtered image;
perform a binarization processing on the filtered image, and obtain the preprocessed image.

12. The electronic device of claim 10, wherein the processor is further caused to:
determine feature points having same pixel values as target feature points, and calculate a distance between any two target feature points;
generate a plurality of first detection frames by connecting two selected target feature points from the determined feature points, wherein a distance between the two selected target feature points is smaller than a preset distance threshold;
determine a plurality of second detection frames from the plurality of first detection frames;
calculate shape similarities between the plurality of second detection frames and a preset shape;
determine the target detection frame from the plurality of second detection frames, with the target detection frame corresponding to a highest shape similarity from the calculated shape similarities.

13. The electronic device of claim 10, wherein the processor is further caused to:
generate a plurality of third detection frames based on the plurality of feature points through a Hough Circle Transform;
select the target detection frame from the plurality of third detection frames based on preset filtering criteria.

14. The electronic device of claim 10, wherein the processor is further caused to:
determine parameter information of the target detection frame;
calculate the first size based on the parameter information.

15. A non-transitory storage medium having stored thereon at least one computer-readable instructions, which when executed by a processor of smart glasses, causes the processor to perform a method of monitoring eye fatigue, wherein the method comprises:
collecting an eye image of a user at a first moment and an eye image at a second moment, wherein the first moment is earlier than the second moment;
determining a first size of a pupil in the eye image at the first moment, and determining a second size of a pupil in the eye image at the second moment;
in response that an absolute difference between the second size and the first size is greater than a preset threshold, collecting an eye image of the user at a third moment, wherein the third moment is later than the second moment; and
in response that an absolute difference between a third size of a pupil in the eye image at the third moment and the second size is greater than the preset threshold, determining that the eye of the user is in a fatigued state.

16. The non-transitory storage medium of claim 15, wherein determining the first size of the pupil in the eye image at the first moment comprises:
acquiring a preprocessed image by pre-processing the eye image at the first moment;
determining a plurality of feature points by performing an edge detection on the preprocessed image;
determining a target detection frame based on the plurality of feature points; and
calculating the first size based on the target detection frame.

17. The non-transitory storage medium of claim 16, wherein acquiring the preprocessed image by pre-processing the eye image at the first moment comprises:
generating a grayscale image by performing a grayscale processing on the eye image at the first moment;
removing noises from the grayscale image by using a preset filter, and obtaining a filtered image;
performing a binarization processing on the filtered image, and obtaining the preprocessed image.

18. The non-transitory storage medium of claim 16, wherein determining the target detection frame based on the plurality of feature points comprises:
determining feature points having same pixel values as target feature points, and calculating a distance between any two target feature points;
generating a plurality of first detection frames by connecting two selected target feature points from the determined feature points, wherein a distance between the two selected target feature points is smaller than a preset distance threshold;
determining a plurality of second detection frames from the plurality of first detection frames;
calculating shape similarities between the plurality of second detection frames and a preset shape;
determining the target detection frame from the plurality of second detection frames, with the target detection frame corresponding to a highest shape similarity from the calculated shape similarities.

19. The non-transitory storage medium of claim 16, wherein determining the target detection frame based on the plurality of feature points comprises:
generating a plurality of third detection frames based on the plurality of feature points through a Hough Circle Transform;
selecting the target detection frame from the plurality of third detection frames based on preset filtering criteria.

20. The non-transitory storage medium of claim 16, wherein calculating the first size based on the target detection frame comprises:
determining parameter information of the target detection frame;
calculating the first size based on the parameter information.
